(19) United States
(12) Patent Application Publication
(10) Pub. No.: US 2005/0054615 A1
(43) Pub. Date: Mar. 10, 2005

(54) Calmodulin independent activation of nitric oxide synthase

(75) Inventors: John C. Salerno, Averill Park, NY (US); Susan M. E. Smith, Averill Park, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Rensselaer Polytechnic Institute, Troy, NY (US)

(21) Appl. No.: 10/855,795

(22) Filed: May 26, 2004

Related U.S. Application Data
(63) Continuation-in-part of application No. PCT/US03/35570, filed on Nov. 7, 2003.
(60) Provisional application No. 60/424,653, filed on Nov. 7, 2002.

(51) Int. Cl. A61K 31/675

(52) U.S. Cl. 514/81; 544/244

(57) ABSTRACT

Methods for activation of a constitutive nitric oxide synthase are described, as are agents which activate a constitutive nitric oxide synthase (e.g., NADPH analogs), methods of identifying such agents, and methods of treatment of diseases or conditions associated with nitric oxide production by a constitutive nitric oxide synthase, by administering an agent which activates the constitutive nitric oxide synthase.

### RELATED APPLICATIONS

This application is a continuation-in-part of International Application No.

PCT/US03/35570, which designated the United States and was filed on Nov. 7, 2003, published in English, which claims the benefit of U.S. Provisional Application No. 60/424,653, filed Nov. 7, 2002. The entire teachings of the above applications are incorporated herein by reference.

### BACKGROUND OF THE INVENTION

Nitric oxide (NO), a small molecule which is highly toxic at moderate concentrations, is a key messenger in mammalian physiology. NO is produced in humans by related enzymes which comprise the nitric oxide synthase (NOS) family.

Two of these enzymes, endothelial NOS (eNOS) and neuronal NOS (nNOS), are constitutively expressed (the “constitutive” NOS isoforms, or cNOS); the third, immune NOS, is inducible.

Endothelial NOS (eNOS) produces NO which controls vascular tone (hence blood pressure), dilates the airways, and controls numerous processes dependent on local dilation of blood vessels, such as gas exchange in lungs, penile erection, and renal function. Brain or neuronal NOS (bNOS or nNOS) produces NO which functions as a neurotransmitter. It is implicated in neural potentiation and bran development, and also controls peristalsis in the gut. eNOS and nNOS are constitutive enzymes controlled by intracellular calcium and the regulatory protein, calmodulin (CaM). The general control mechanism in these constitutive NOS isoforms is the calcium/calmodulin dependent switching of interdomain electron transfer, which requires the CAM binding site and the autoinhibitory element of the FMN binding domain (Salerno, J. et al., J. Biol. Chem. 272:29769 (1997)) and which correlates with the presence of additional sequence elements in the FAD binding domain and C terminal. Removal of the C terminal tail has been reported to produce a truncated, constitutively active eNOS (Roman, L. J. et al., Chemical Reviews 102:1179 (2002)). Recently, electron transfer through the reductase domains of NOS has been reported to be calmodulin independent in the absence of NADPH in rapid kinetics experiments (Daff, S. et al., Nitric Oxide 6:366 (2002)).

### SUMMARY OF THE INVENTION

The present invention pertains to agents which activate a constitutive nitric oxide synthase (eNOS, nNOS) by inhibitor displacement, such as by displacing NADP+/NADPH; by having binding domain overlap on the nitric oxide synthase with NADPH; by filling the adenine portion of the pyridine nucleotide binding site on the nitric oxide synthase without initiating inhibition of electron transfer; or by preventing binding of NADPH to the nitric oxide synthase, without itself inducing a locked conformation. Representative agents which activate a constitutive NOS by inhibitor displacement include NADPH analogs (e.g., 2′ AMP; 5′ AMP; 2′5′ADP; ADP, ATP); and agents comprising heterocyclic aromatic rings having one or more side chain(s) upon which one or more negatively charged atom(s) or molecule(s) is attached. In preferred embodiments, the side chain of the heterocyclic aromatic ring is a ribose, and the negatively charged molecule is attached at the 2′ position, the 5′ position, or both the 2′ and 5′ positions of the ribose. The invention also pertains to methods of identifying agents that modulate activity of a constitutive nitric oxide synthase, by assaying the ability of the agents to displace an inhibitor (e.g., NADP+/NADPH); as well as to methods of identifying agents that modulate activity of a constitutive nitric oxide synthase, by assaying the ability of the agents to compete with NADPH for binding to the constitutive nitric oxide synthase. The invention additionally pertains to methods of altering activity of a constitutive nitric oxide synthase, by contacting the nitric oxide synthase with an agent as described. In one embodiment, the agent is incorporated into a biocompatible carrier, and/or is a component of an implantable medical device, such as a stent. The invention further pertains to methods of treating a disease modulated by production of nitric oxide by a constitutive nitric oxide synthase in an individual, by administering to the individual an effective amount of an agent as described.

The agents and methods of the invention provide a means for activating a constitutive NOS in a manner that differs from other previously known methods of activating constitutive NOS, and thereby broaden the scope of available activators for these important enzymes as well as the scope of therapeutics for NO-mediated diseases and conditions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram depicting the modular structure of nitric oxide synthase (NOS).

FIG. 2 is a schematic diagram depicting the folding of NOS.

FIG. 3 depicts the structures of noose reductase domains of NOS and P450 reductase showing hinge region and FMN, FAD, and NADPH binding domains. NOS trace is in cyan with beta regions in red. Hinge subdomain small insertion, adjacent to CaM binding site, is in white.

FIG. 4 depicts the linkage between ligand binding and conformation that is provided by the extended linker joining the FMN and FAD domains. It interacts successively with the FMN domain and calmodulin before forming the initial strand of the FAD binding beta barrel and a short loop which forms key cross domain H bonds with NADPH.

FIG. 5 depicts the cross-domain interaction of R1010 nNOS/R778 eNOS cognate in loop with NADP adenine. Barrel domain (FAD binding) is on right, sheet domain (NADPH binding) on upper left. R1010 side chain shown in red.

FIG. 6 depicts details of NADPH binding site and interaction with R1010 cognate. R1010 side chain shown in red.

FIG. 7 depicts an alignment of the gene and amino acid sequences of rat nNOS, bovine eNOS, and mouse iNOS in the small insertion region showing the position and extent of the insertion in the constitutive isoforms relative to iNOS. Apparent frame shifts have destroyed all similarity at the amino acid level between eNOS and nNOS; several slightly different equivalent alignments are possible. Although the presence of the small insertion (SI) correlates well with Ca+2/CaM control and the presence of an autoinhibitory insertion (AI) elsewhere in the sequence, there is no homology at the amino acid level. The amino acid sequence of human NADPH P450 oxidoreductase (CPNR) is shown for comparison.

FIG. 8 depicts the UV-visible absorbance spectra of wild type NOS enzymes and mutants, showing contributions from the heme and flavin cofactors. Spectra are shown for wild type nNOS and the G1074Y and G1074D mutants; compare the heme Soret band near 400 nm with the FAD and FMN features in the 450-500 nm region.

FIG. 9 depicts Cytochrome c reductase activity of wild type nNOS and nNOS mutants, in the absence of calcium and calmodulin.

FIG. 10 depicts Cytochrome c reductase activity of wild type nNOS and nNOS mutants, in the presence of calcium and calmodulin.

FIG. 11 shows UV-visible absorbance spectra of wild type NOS enzymes and mutants, showing contributions from the heme and flavin cofactors. Spectra are shown for wild type eNOS and the corresponding G841Y and G841D mutants; both mutants are almost completely heme free.

FIG. 12 shows an SDS PAGE gel of partially purified wild type NOS preparations and selected mutants, showing the effects of proteolysis on the enzymes during expression in E. coli BL21. Lanes one through four contain nNOS mutants: lane one, nNOS Y879S (FMN shielding residue) mutant; lane two, nNOS—GVIS mutant; lane three, TALYVIS (G1074Y) mutant; lane four, TALDVIS (G1074D) mutant. Lane five has molecular weight markers at 97.4, 66.2, and 45 kD. Lane six contains wild type iNOS and lane seven is a repeat of the nNOS Y879S mutant. Lane eight is wild-type eNOS(SPGGPPP); lanes nine through eleven are eNOS mutants. Lane nine, eNOS SPGPPP (G841 deletion) mutant; lane ten, eNOS SPGYPPP (G841Y) mutant; lane eleven, eNOS SPGDPPP (G841D) mutant.

FIG. 13 depicts results from plate reader Greiss assays, demonstrating that the titration of eNOS with 2′AMP in the presence of ‘fully’ activating levels of Ca+2/CaM produced additional activation with apparent Kd of ˜1 mM, suggesting the effect is due to competition of ligands with similar binding constants (NADPH and 2′AMP).

FIG. 14 depicts activation of electron transfer wild type cNOS isoforms by ATP in Tris buffer in the absence of calmodulin.

FIG. 15 depicts inhibition of INOS by ATP in Tris buffer in the absence of calmodulin.

### DETAILED DESCRIPTION OF THE INVENTION

The present invention pertains to the discovery of the ability of an NADPH analog to hyperactivate NOS, particularly eNOS. As described herein, NOS isoforms are regulated by Ca+2/calmodulin (CaM). However, abnormalities were noted in the activity of certain cNOS preparations. It was observed that affinity chromatography purified eNOS was active in the absence of CaM prior to dialysis. Surprisingly, removal of eluent 2′ AMP, an NADPH analog, removed the CaM independent activity and restored calcium/CaM control. Other potential causes for the CaM independent activation (buffer, pH, high ionic strength) were eliminated (although all these factors have secondary effects on activity). 2′AMP is a competitive inhibitor in other enzymes with respect to NADPH, suggesting that displacement of NADPH/NADP+ from its binding site produced a state in which the reductase domains were competent to support catalysis by electron transfer. Without being bound by a particular theory of the mechanism, it is believed that activation occurs because NADPH is prevented from binding to the nitric oxide synthase, without induction of a locked conformation. Partial inhibition of electron transfer into eNOS or nNOS from NADPH may not be important because NOS reduction is three orders of magnitude faster than transfer of electrons to the oxygenase domain.

The results were confirmed and extended using microtiter Griess activity assays of NO production in 96 well plates. 2′AMP not only activated cNOS in the absence of calmodulin (particularly under certain conditions, e.g., in Tris buffer), but in general produced hyperactivation of eNOS in the presence of calcium/CaM. Specifically, when the initial concentrations of NADPH and 2′AMP were equal, the activity of eNOS was 2-3 times that of eNOS activated by CaM alone. These effects appeared to correlate with changes in the optical spectra of the enzyme in the charge transfer region and with shifts in the FAD optical bands at 450 and 485 nm.

These results are supported by the hypothesis that NADPH/NADP+produce a “conformational lock” in cNOS that inhibits electron transfer (Daff, S. et al., Nitric Oxide 6:366 (2002)). Inhibitors which fill the adenine portion of the pyridine nucleotide binding site can potentially activate electron transfer by displacement of NADPH/NADP+. Since NADPH reduction of cNOS, particularly eNOS, is not close to rate limiting, a wide range of inhibitor concentrations can be tolerated before inhibition of electron donation by NADPH becomes more important than activation by removal of the charge transfer complex.

As a result of these discoveries, agents can be identified which can be used to activate constitutive NOS isoforms. The activation occurs by inhibitor displacement, such as by displacement of the reductant NADP+/NADPH. For example, in one embodiment, the agent can be an agent which has binding domain overlap on the NOS enzyme with NADPH (e.g., an agent which fills the adenine portion of the pyridine nucleotide binding site), but which does not initiate inhibition of electron transfer (as does NADPH). In another embodiment, the agent can be an agent which prevents binding of NADPH. In certain embodiments, the agent is an analog of the adenosine part of NADPH; in another particular embodiment, the agent is an analog having a phosphate at the 2′ position. Representative NADPH analogs include 2′AMP; 5′ AMP; 2′5′ADP; ADP; ATP. In certain other embodiments, the agent comprises a heterocyclic aromatic ring, the ring having one or more side chain(s) upon which one or more negatively charged atom(s) or molecule(s) is attached. It is believed that the heterocyclic aromatic ring will have a stacking interaction with the NOS enzyme structure at the aromatic amino side chains of NOS. The side chain(s) (e.g., a ribose or other linker) of the heterocyclic aromatic ring has attached to it, one or more phosphate(s) or other negatively charged atom(s) or molecule(s). In preferred embodiments, the side chain is a ribose, and the negatively charged molecule is attached at the 2′ position, the 5′ position, or both the 2′ and 5′ positions of the ribose.

In addition, methods are now available for activating constitutive NOS activity by administration of the agents, as well as methods for treating diseases or conditions associated with NO production by constitutive NOS isoforms by administration of the agents. The invention additionally pertains to use of the agents, as described herein, for the manufacture of a medicament for the treatment of diseases or conditions associated with NO production by constitutive NOS isoforms.

Isolating and Identifying New NOS Activators and Inhibitors

Based on the discoveries described herein, it is now possible to identify agents which modulate (increase or decrease) the activity of a constitutive NOS. Agents which “increase” the activity are those which activate or promote the activity of the NOS. Agents which “decrease” the activity are those which inactivate, interfere with, minimize or prevent the activity of the enzyme. Agents of the invention can modulate NOS activity independently of calmodulin (CaM) activation; that is, whether or not CaM is associated with the NOS, it is the agent, rather than the CaM, that modulates the NOS activity.

In the methods of the invention, agents of interest (the “test agent”) are assessed for an ability to modulate the activity of a constitutive NOS. Screens for agents are performed in a manner so that it can determined whether the test agent is competing with NADPH for interaction with NOS (e.g., displacing the inhibitor, such as displacing NADPH/NADP+). Thus, an excess of NADPH is not desirable; rather, the amount of NADPH in assays to screen for agents of interest should be varied, in order to facilitate detection of competition between the test agent and NADPH.

Bearing in mind these considerations, assays can be used to determine whether an agent modulates NOS activity. A sample of the agent to be tested (the “test agent”) is contacted with a sample of a constitutive NOS, thereby generating a test sample (herein referred to as a “synthase sample”). After incubation of the synthase sample under conditions appropriate for activity of the enzyme (e.g., in the presence of NADPH), the level of NOS activity is measured by standard methods (e.g., by measurement of production of NO). The level of NOS activity is then measured and compared to the amount of activity in a control sample under the same conditions but in the absence of the test agent. If the level of activity in the synthase test sample is different from the level of activity of a control sample of the NOS under the same conditions but in the absence of the test agent, then the agent modulates the activity of the NOS. Similar assays can be used to determine whether an agent modulates the activity of one constitutive NOS isoform without modulating the activity of other constitutive NOS isoform, by comparing the level of activity of each isoform in the presence of the agent. Additional description of assays for determining whether an agent modulates NOS activity can be found, for example, in U.S. Pat. No. 6,150,500, the entire teachings of which are incorporated herein by reference.

Agents

Agents of the invention include agents that activate a constitutive nitric oxide synthase (NOS) by inhibitor displacement, such as by displacement of the reductant NADP+/NADPH. For example, in one embodiment, the agent can be an agent which has binding domain overlap on the NOS enzyme with NADPH (e.g., an agent which fills the adenine portion of the pyridine nucleotide binding site), but which does not initiate inhibition of electron transfer (as does NADPH). In another embodiment, the agent can be an agent which prevents binding of NADPH. Representative agents include NADPH analogs. In one particular embodiment, the agent is an analog of the adenosine part of NADPH; in another particular embodiment, the agent is an analog having a phosphate at the 2′ position. Representative NADPH analogs include 2′AMP; 5′ AMP; 2′5′ADP; ADP; ATP. In other embodiments, the agent comprises a heterocyclic aromatic ring, the ring having one or more side chain(s) (e.g., ribose or other linker) upon which one or more negatively charged atom(s) or molecule(s) (e.g., phosphate) can reside. In preferred embodiments, a side chain of the heterocyclic aromatic ring is a ribose, and a negatively charged molecule is attached at the 2′ position, the 5′ position, or both the 2′ and 5′ positions of the ribose.

Methods of Altering NOS Activity

The agents that activate a constitutive NOS, as described herein, can be used to activate a constitutive NOS isoform. To activate the constitutive NOS, the NOS of interest (eNOS or nNOS, or both) is contacted with an agent as described herein, under conditions for interaction between the agent and the NOS of interest. More than one agent can be used concurrently, if desired.

Methods of Treatment

The agents that activate a constitutive NOS can also be used to activate the constitutive NOS isoform in vivo. In a preferred embodiment, the agent is used to activate a constitutive NOS isoform in a mammal, such as a human, in order to treat a disease or condition associated with NO production. The term, “treatment” as used herein, refers not only to ameliorating symptoms associated with the disease or condition, but also preventing or delaying the onset of the disease or condition, and also lessening the severity or frequency of symptoms of the disease or condition.

For example, in certain methods of the invention, one or more agent(s) that activate a constitutive NOS (e.g., an agent described herein) is administered to an individual. The agent can be administered in dosage formulations containing conventional, non-toxic, physiologically-acceptable carrier(s) or excipient(s) The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral, intranasal, subcutaneous, rectal, buccal, vaginal, intraurethral, by inhalation spray, or via an implanted reservoir. Other suitable methods of introduction can also include rechargeable or biodegradable devices, particle acceleration devises (“gene guns”) and slow release polymeric devices. In one embodiment of the invention, the composition is incorporated into a biocompatible carrier (e.g., a biopolymer or other coating), and/or is a component of an implantable medical device (e.g., a stent). A composition that is incorporated into a biocompatible carrier, for example, is suspended mixed with, or encapsulated, or otherwise integrated into the biocompatible carrier. The composition can be incorporated into a biopolymer which forms part of, or is coated onto or included within or on, an implantable medical device. An implantable medical device refers to a medical device designed to be inserted or otherwise placed inside the body of a patient, or otherwise in contact with an internal part of a patient. The biocompatible carrier, if associated with or a component of a device, is designed such that the composition is released (delivered) after implantation of the device. In a particularly preferred embodiment, the implantable medical device is a stent. Representative biocompatible carriers and coatings for stents, as well as stents using such carriers and coatings, are described, for example, in U.S. Pat. Nos. 6,716,445; 6,713,119; 6,702,805; 6,656,162; 6,530,951; 6,299,604; and 6,096,070. These patents are incorporated herein by reference in their entirety.

The pharmaceutical compositions of this invention can also be administered as part of a combinatorial therapy with other therapeutic agents. The composition can be formulated in accordance with the routine procedures as a pharmaceutical composition adapted for administration to human beings or other mammals of interest. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The therapeutically effective amount can be administered in a single dose, or a series of doses separated by appropriate intervals, such as hours, days, or weeks. The term “single dose,” as used herein, can be a solitary dose, and can also be a sustained release dose, such as by a controlled-release dosage formulation (e.g., in a biocompatible carrier as described above) or a continuous infusion. Other drugs can also be administered in conjunction with the agent, and more than one agent that activates a constitutive NOS can be administered at the same time.

Therapeutic targets for constitutive NOS activation, and particularly eNOS activation, include diseases or conditions modulated by production of nitric oxide by the cNOS. An agent that activates a constitutive NOS is administered in order to treat the condition modulated by production of nitric oxide by the cNOS. For example, agents to activate NOS can increase NO production and thereby aid in the treatment of hypertension. Also, NO produced by eNOS is a regulatory factor in secretory processes (e.g., insulin production), so disease processes associated with insulin (e.g., diabetes) can be targeted. Regulation of perfusion in the lung and airway tone are also eNOS controlled, so that lung conditions (e.g., emphysema, asthma) can be targeted. In another embodiment, enhancement of angiogenesis can be performed. Stimulation of eNOS will facilitate development of collateral circulation in individuals in need thereof (e.g., individuals having had myocardial infarction or ischemic disease, limb reattachment, or other need for angiogenesis). Furthermore, because, nNOS controls processes such as peristalsis and is involved in signaling in skeletal muscles, it may be involved in signaling at connections between the nervous system and secretory systems, yielding additional targets for use of the agents herein. In certain preferred embodiments of the invention, an agent that activates a constitutive NOS is administered in order to treat a condition modulated by production of nitric oxide by the cNOS (e.g., hypertension, atherosclerosis, diabetes, emphysema, or acute asthma). In another preferred embodiment of the invention, an agent which activates a cNOS can be used as a means for treating male erectile dysfunction; in one embodiment of treating male erectile dysfunction, the agent is administered intraurethrally to limit systemic side effects.

Discussion

The discoveries described herein have enabled a better understanding of the activation of NOS and have furthered investigation of the role of calmodulin in the control mechanisms of NOS. An analysis of the role of calmodulin, particularly with regard to mutant forms of NOS, underscores the surprising nature of the calmodulin-independent activation of the invention.

Nitric Oxide Synthases (NOS) are an enzyme family producing nitric oxide from arginine in a reaction requiring 2 mol oxygen and 3/2 mol NADPH per mol NO. They are large modular proteins with heme, tetrahydrobiopterin, FAD and FMN prosthetic groups (Alderton, W. K., et al., Nitric oxide synthases: structure, function and inhibition Biochem. J. 357: 593-615(2001); Ghosh, D. K. and Salerno J. C., Nitric oxide synthases: domain structure and alignment in enzyme function and control. Front. Biosci. 8: 193-209,2003). Constitutive nitric oxide synthases (cNOS) include neuronal (nNOS) and endothelial (eNOS) isoforms, and are regulated by Ca+2/calmodulin. The third mammalian isoform, iNOS, is induced during immune response by factors including cytokine. It binds calmodulin at all physiological calcium concentrations, producing cytotoxic levels of NO as part of immune response.

Mammalian cNOS is controlled primarily through regulation of the delivery of electrons derived from NADPH to the catalytic site. Recent work shows that each NOS is a homodimeric polypeptide and is comprised of a N-terminal heme containing catalytic oxygenase domain and a C-terminal reductase domain with an intervening calmodulin (CaM) binding sequence (id). Reducing equivalents flow through reductase domains, homologous to P450 reductase (NCPR), which include an NADPH binding domain, and FAD binding domain, and a FMN binding domain. The primary regulation appears to be the reduction of heme by FMN, but electron transfer from FAD to FMN may also be affected. In the absence of Ca+2/calmodulin, electron transfer from FMN to heme typically does not occur even on a time scale of hours.

Control of the signal generating constitutive isoforms (cNOSs) is exerted primarily by Ca+2/calmodulin (Salerno, J. C., et al., J. Biol. Chem., 272, 29769 (1997)). Inducible NOS (iNOS), due to its tight coupling to CaM under basal Ca+2 levels, is notably distinguished from cNOSs by its sustained high output NO production even at low levels of calcium (Alderton, W. K., et al., Biochem. J. 357: 593-615(2001); Ghosh, D. K. and Salerno J. C., Front. Biosci. 8: 193-209,2003).

The head to tail arrangement of the NOS dimer, in which the reductase unit of one monomer reduces the oxygenase domain of the other, suggests that several additional surfaces may play a role in catalysis and control. Inspection of the oxygenase dimer indicates that an oxygenase domain surface on the opposite face of each monomer from the reductase domain which supplies it with electrons faces a bound calmodulin. Residues in this region include the highly conserved C terminal edge of the oxygenase domain preceding the canonical CaM binding sequence, which is remote from the catalytic site, and two loops containing (in eNOS) residues around S78 and Q257. In addition to this ‘intramonomer’ CaM interaction surface, a second ‘intermonomer’ CaM interaction surface is likely to exist at the edge of the interface with the FMN binding domain. Loops from the other monomer contribute residues to all these surfaces; one such loop contains E388 in eNOS.

Other surfaces may interact with CaM; they include the opposite edge of the FMN binding domain from the FMN binding site, which carries the AI, and subdomain regions adjacent to it, including the SI. All these surfaces contain residues which are candidates for specific interactions with CaM. In addition, modification of CaM itself is a powerful tool for the study of interactions with NOS. A wide variety of naturally occurring calmodulins and related EF hand proteins are available differing in their ability to activate NOS isoforms. In addition, a large number of calmodulin mutants and constructs making use of the repeating EF hand structure have been made, and it is easier to mutate specific residues on the surface of calmodulin than to produce NOS mutants.

Structural information can provide clues to the electron transfer control mechanism. FMN binding domain orientation places FMN adjacent to the FAD isoalloxazine in the neighboring domain (Wang, M., D. et al., 1997. Proc Natl Acad Sci USA. 94:8411-6). Bound CaM, at the β-sheet edge opposite FMN, is thus remote from reductase cofactors (Salerno, J. C., et al., 1997, J. Biol. Chem. 272:29769-77). It was determined that an insertion in this domain is a control element (the AI) and provided evidence for an autoinhibitory role (id.). AI occurrence in FMN binding domains correlates with Ca+2/CaM control. The AI lies in an α>β loop on the β sheet edge opposite FMN, but directly adjacent to CaM. This arrangement has been confirmed by several groups, which have produced constructs (e.g., AI deletions of eNOS and nNOS) which confirm its autoinhibitory role.

Mutants in a small insertion (the SI) in the FAD subdomain correlate with the presence of an AI in a large number of NOS isoforms in organisms ranging from primitive eukaryotes to mammals. The effects of SI mutations on control and activity are complex. The SI is spatially adjacent to bound CaM and AI, as predicted by the PI and confirmed crystallographically. Removal of AI or SI can produce an iNOS-like enzyme with high activity and low Ca+2 requirements, but which still requires CaM. Existing evidence indicates that CaM, AI, and SI interact directly, forming a triad of control elements.

Data from several laboratories show that the tail region extending from NADPH binding domain to C terminus is involved in the inhibition of uncontrolled electron transfer. This information includes N terminal truncation and serine mutation/phosphorylation experiments (e.g., Roman L. J., et al., 2000. J Biol Chem 22;275(38):29225-32; Adak S., et al., 2001. J Biol Chem 12;276(2):1244-523).

Recently, additional information has been provided in the form of FAD shielding residue mutants (Adak S, et al., 2002,. Proc. Natl. Acad. Sci. 99(21):13516-21), and kinetics experiments have revealed a ‘conformational lock’ dependent on NADPH (Craig DH, et al., 2002.J Biol Chem 13;277(37):33987-94). These results implicate the equilibrium between the NADPH/FAD charge transfer complex and alternative bound NADPH configurations in control of domain alignment.

Elements involved in C terminal restriction of electron flow are spatially remote from the CaM/AI/SI triad. C terminal modifications are often characterized by high electron transfer activity in the absence of CaM with low (10%) rates of NO synthesis. CaM evokes wild type behavior, suppressing uncoupled electron transfer, and allowing up to 50% of wild type levels of NO synthesis. C terminally truncated NOSs in particular lose specificity (are ‘uncoupled’) in the absence of CaM, but CaM restores most of the wild type character to these mutants.

Other CaM control mechanisms incorporate autoinhibitory elements interacting directly with CaM (Daff S., et al.,1999, J Biol Chem 22;274(43):30589-95). NOS control complexity has several sources, including suppression of uncoupled electron transfer/superoxide formation, the probable genesis of the C terminal mechanism. AI and SI development is directly related to CaM control. Mammalian isoforms also incorporate additional inputs involving covalent modifications and multiprotein complex formation.

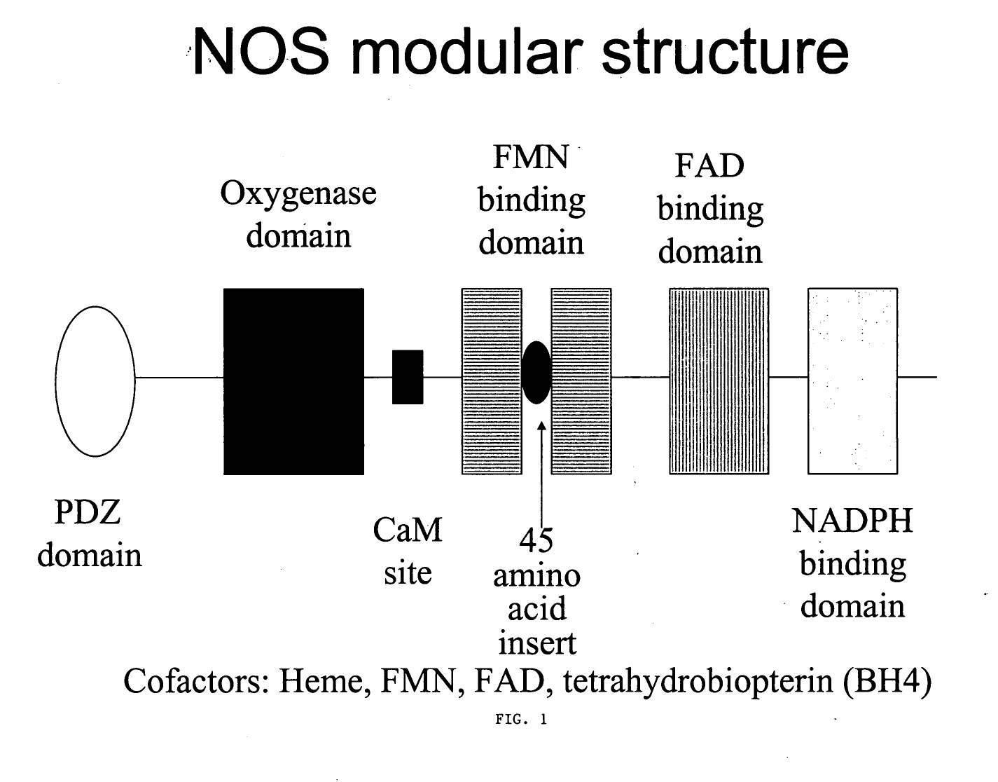

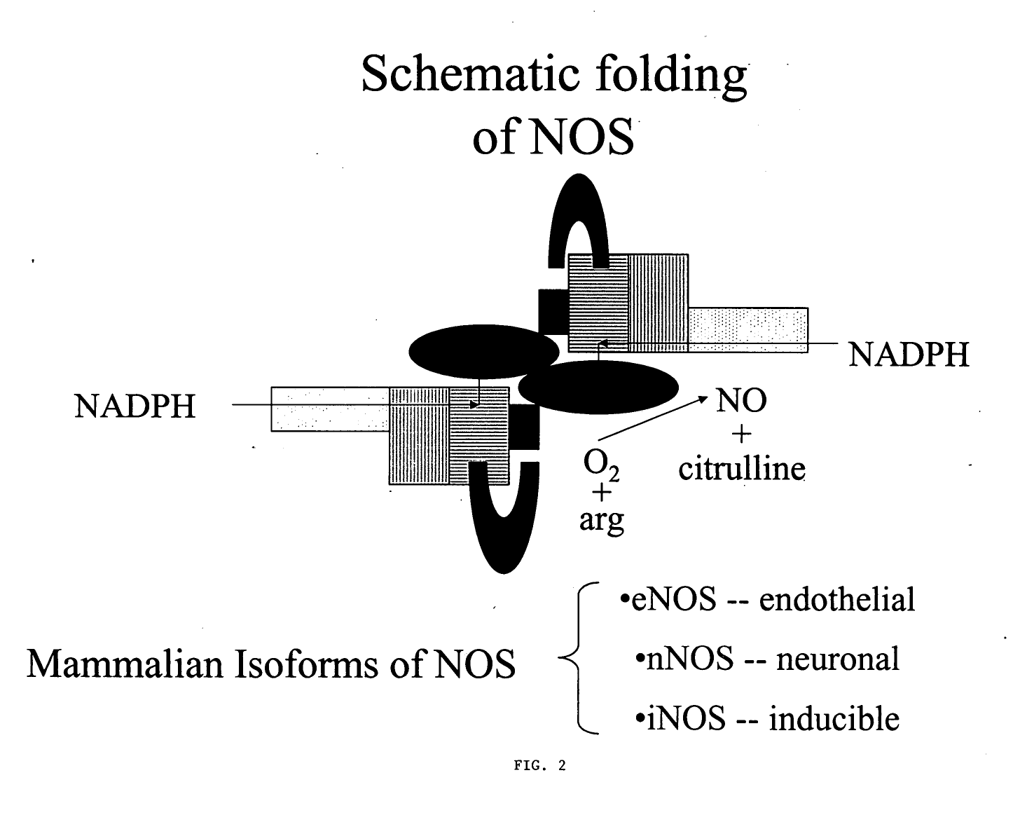

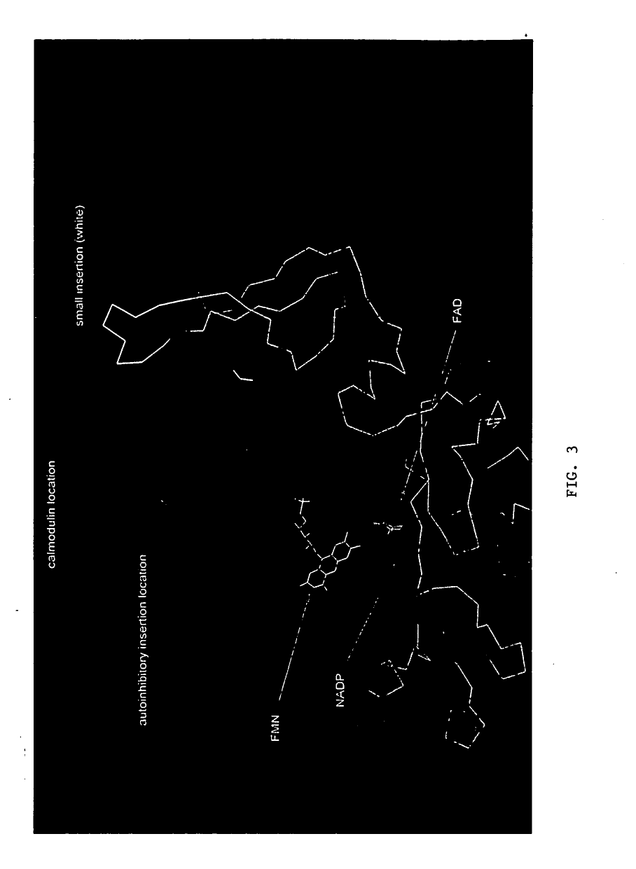

The structural organization of NOS is summarized in FIGS. 1 and 2; the domains are carried on a single polypeptide, two of which form a homodimer as described above, with the oxygenase domain toward the N terminal and the reductase domains on the C terminal side of the calmodulin binding site. FIG. 3 shows a view of the structure of the reductase domains. Calmodulin binds directly adjacent to the FMN binding domain, and is placed to interact directly with the AI, a 40-50 residue insertion in the FMN domain of cNOSs, and with the SI, a small (6-7 residue) insertion in the hinge subdomain. In FIG. 3, the structure of cytochrome P450 oxidoreductase (Wang, M., et al., (1997) Proc Natl Acad Sci USA 94 (16): 8411-8416) is overlaid with the structure of a two domain nNOS construct, including the FAD and NADPH binding domains and the hinge subdomain (Zhang J., et al., (2001) J. Biol. Chem. 276(40):37506-13). The small insertion is shown in white; it is adjacent to the edge of the FMN binding domain opposite the FMN binding site, and is directly adjacent to bound calmodulin and the AI. In nNOS the sequence immediately preceding the SI contains the triplet LEE, corresponding to LDE in iNOS and NADPH P450 oxidoreductase and LEK in eNOS. The second acidic residue in the figure, corresponding to E352 in NADPH P450 oxidoreductase and E1068 in rat nNOS, is marked in deep red in both cases. Immediately following the SI is a triplet NKK in NADPH P450 reductase and NWK in nNOS. The serine residues, S354 and S1077 in NADPH P450 oxidoreductase and nNOS respectively, are marked in yellow. This clarifies the relative positions of cognate structures (e.g., the AI and CaM binding site) surrounding the SI region in the two proteins.

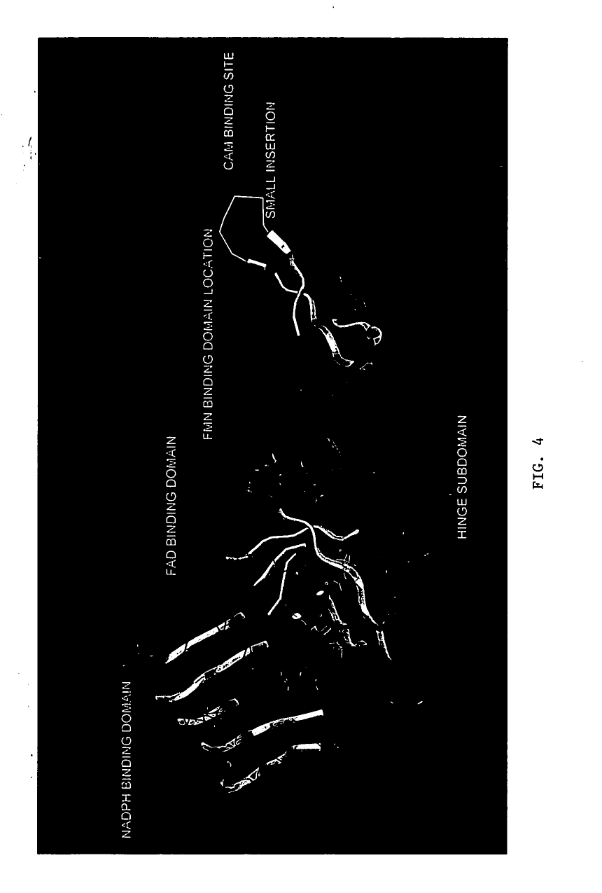

FIG. 4 shows a view of the FAD and NADPH binding domains and the hinge subdomain; the FMN binding domain has been omitted for the sake of clarity. On the right hand side of the figure the beat pair carrying the SI forms a three stranded beta sheet with the linker connecting the FMN and FAD domains. The linker is shown in cyan except for the beta region, shown in orange. This provides a strong connection to bound calmodulin, since the FMN domain end of the linker is directly adjacent to bound CaM and the SI, carried on the interacting beta pair, apparently evolved specifically to interact with the CaM site of cNOSs. A span of ˜20 residues connected the beta region of the linker with its termination in the initial beta barrel strand of the FAD binding domain; this include a rigid short helix H bonded to the initiating region of the barrel strand. Immediately after forming the barrel strand, the polypeptide forms a loop carrying residues involved in cross-domain interactions with NADPH, including R1010 in rat nNOS(R1015 in human nNOS and R778 in bovine eNOS). This single relatively short connector links the CaM binding site and the adjacent FMN binding domains with the FAD and NADPH binding sites, providing a rationale for the coupling between CaM binding and NADPH binding which lies at the heart of activation by NADPH analogs.

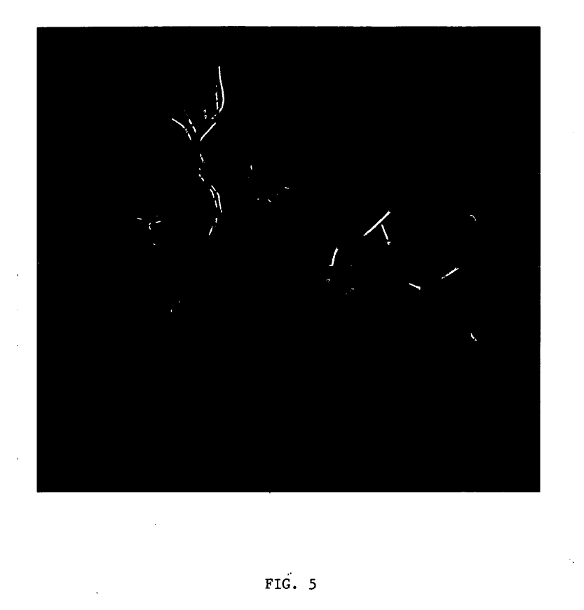

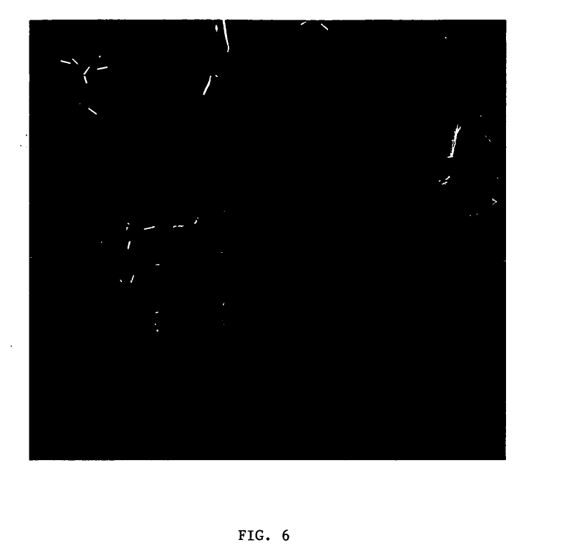

The interactions between R1010 and its cognates and NADPH adenine are more easily seen in the expanded scales of FIGS. 5 and 6. The terminal e (epsilon) amino groups of the arginine side chain form three hydrogen bonds with the adenine ring system.

An assessment of NOS mutants was also performed to investigate control mechanisms.

All chemicals used for purification were obtained from Sigma Chemical Co. The genes for eNOS and nNOS were gifts from Professor B.S.S. Masters (University of Texas Health Science Center at San Antonio); the iNOS expression system was the gift of Professor Dipak Ghosh (Duke University and VA Medical Center). GroELS plasmid was provided by Dr. Anthony Gatenby (Dupont).

Expression and purification of wild type and mutant bovine eNOS and rat nNOS. Expression and purification of bovine eNOS and rat nNOS were performed using procedures similar to those previously described (Martasek, P.,et al., (1996) Biochem Biophys Res Commun. 219(2); 359-65). Transformed cells were broken with a French press, and after centrifugation to remove cell debris the supernatant was loaded on a 2′5′ADP affinity column, washed, and eluted with 2′AMP as described (id). High purity preparations can be obtained with a size exclusion step; we used a Superose 6 HR 10/30 column (Pharmacia Biotech AB, Uppsala, Sweden), flow rate 0.4 mL/min, buffer composition −50 mM Tris-HCl, pH 7.5, 0.1 mM EDTA, 1 mM-mercaptoethanol, 100 mM NaCl, 10% glycerol (vol/vol). During the purification procedure BH4 or L-arginine were added after elution from the affinity column. Enzyme sample concentrations were determined on the basis of heme concentration, except in heme free preparations, where the flavin concentration was measured. UV-visible absorbance spectra were recorded on an Aminco DW-2000 spectrophotometer.

Nitric Oxide production was assayed using the Griess assay as adapted for microtiter plates (Gross, S. S. (1996) Methods Enzymol. 268:159-68); calcium dependence was measured using EDTA as a Ca+2 buffer system. NOS isoforms were routinely assayed with different buffering systems, since eNOS is much more active in MOPS while iNOS and eNOS work well in Tris. NADPH-dependent cytochrome c reduction was measured spectrophotometrically as described by McMillan et al (McMillan, K., et al., (1992) Proc Nat. Acad. Sci. U.S.A. 89, 11141-11145), adapted to 96-well plate microtiter plates. In each well, the 500-uL reaction mixture contained 50 mM Na+ TRIS buffer, pH 7.5, 50 uM EDTA, 50 uM NADPH, 50 uM horse heart cytochrome c, and ˜10 nM of nNOS. Cytochrome c reduction was monitored at 550 nm (ε=2.1×104/M). In Cam/Ca+2 dependence studies, 0.75 uM CaCl₂ and 10 pg/ml calmodulin were added to reaction mixtures. Assays were performed on a SpectraMAX plate reader (Molecular Devices).

Generation of NOS mutants. NOS genes in pCWori+ were mutated using a method we devised employing the Stratagene Quik Change Mutagenesis kit. As suggested by Wang and Malcolm (Wang, W Y & Malcolm, BA (1999) Biotechniques 26(4):680-682), we began by separating the forward and reverse primers, but instead of a single preliminary step with separate primers followed by reversion to the Stratagene protocol, we employ 25 cycles of linear amplification with no additional steps other than to combine and anneal the samples. The removal of the ‘PCR’ steps, which are unproductive and which actually destroy mutant strands by extension, removes the limitations on the separate linear amplification steps and greatly improves the performance of the procedure. In particular, we have obtained a consistent high yield of mutants and a very low background (<5%) of parentals.

After mutagenesis, the products were used to transform XL 10-Gold Ultracompetent E. coli cells from Stratagene. Transformed cells were selected by growth on ampicillin media; clonal colonies were cultured and plasmid DNA was obtained using Qiagen miniprep kits. Mutants were sequenced at the State University of New York at Albany Center for Functional Genomics sequencing facility.

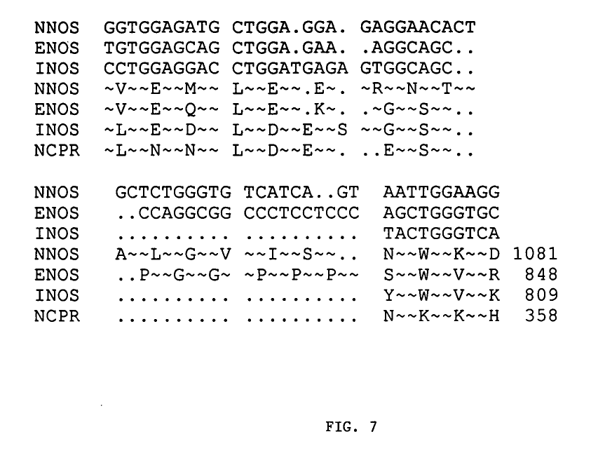

An alignment of the small insertion region in eNOS, nNOS, iNOS, and NADPH P450 oxidoreductase is shown in FIG. 7. Sequence accession numbers are: rat nNOS, GenBank X59949 (Bredt, D. S., et al., (1991) Nature 351, 714-718), bovine eNOS, GenBank M89952 (Lamas, S., et al., (1992) Proc. Natl. Acad. Sci. U.S.A. 89, 6348-6352), murine iNOS, EMBL G198407 (Bredt, D. S., et al., (1991) Nature 351, 714-718; Xie, Q., et al., (1992) Science, 256, 225-228), NADPH P450 reductase, SwissProt P16435 (Hainu, M., et al., (1989) Biochem. 28; 8639-8645). These sequences are part of the hinge subdomain within the FAD binding domain. Alignments of two dozen eukaryotic NOS sequences indicate that corresponding small insertions are a feature of other NOS sequences that contain AI cognates in the FMN binding domain; iNOSs lack this feature. The SI regions of constitutive NOS isoforms of primitive vertebrates and invertebrates resemble nNOS more closely than eNOS. The small insertion in mammalian nNOS is characterized by its hydrophobic character and by a glycine residue that marks a turning point in the path of the backbone. It appears that a centrally located glycine (or glycines) allowing a sharp turn may be the only common SI sequence element.

In order to examine the significance of this residue we constructed several mutants. We reasoned that the standard alanine mutant would not be particularly informative in this case, since Gly and Ala both have small side chains associated with sharp turns. Tyr and Asp were selected as residues that would introduce strongly different character into the region, Tyr by virtue of its large side chain volume and Asp by introducing a negative charge into a hydrophobic region. We preferred the relatively short side chain of acidic residues to the very long positively charged side chains of Arg and Lys, both of which are capable of interacting over a considerable distance.

Mutants of nNOS. The structure of the nNOS region can be seen in FIG. 3, described above. In designing deletion mutants we were influenced by the pattern observed in large multiple sequence alignments of NOS isoforms, by the structures of P450 reductase and nNOS reductase domains shown in FIG. 3, and by structural models of the NOS reductase domains we have constructed over the past ten years using information from these and other solved homologous proteins (e.g., 22). In P450 oxidoreductase (and probably in iNOS), the motif LDEES (see FIG. 7) forms the turn at the end of a series of β pair structures. The insertion of six or seven residues relative to iNOS or P450 reductase causes the corresponding sequence element to assume a different position; it now forms one side of a terminal β hairpin structure. Removal of the entire insertion (corresponding to within one residue to TALGVIS in nNOS and PGGPPP in eNOS) should cause a reversion to the P450 reductase like structure if this is consistent with the structural context. Partial deletions should cause shortening of the β hairpin with potential disruption of the local geometry, since the shorter sequence cannot make the same turn as the wild type parent. Note the large difference in structure between the flanking homologous regions in the two structures overlaid in FIG. 3; this might have less to do with the insertion than with the differences in structurally adjacent regions in the two enzymes shown, and with the absence of the FMN binding domain in the nNOS structure.

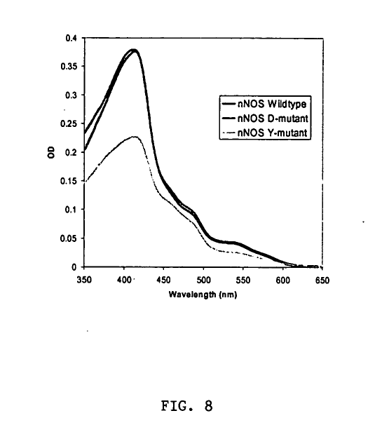

FIG. 8 shows optical spectra of preparations (partially purified by 2′5′-ADP affinity chromatography) of the G1074Y and G1074D rat nNOS mutants as well as wild type enzyme. G1074Y has an abnormal absorbance spectrum reflecting a heme to flavin ratio of 1:4 rather than the 1:2 ratio of wild type enzyme, while the spectrum of G1074D closely resembles that of wild type nNOS. This is most obvious from the relative size of the heme Soret band near 400 nm, indicative of a majority high spin state and a minority low spin state of ferriheme, and the bands near 480 nm from FAD and FMN. We have confirmed the loss of heme by decomposition of the spectra into heme and flavin components. This unusual and somewhat counterintuitive result implies that a mutation in the reductase domains reduced the heme content while leaving the flavin content relatively unchanged. CO difference spectra (not shown) indicate that the ferrous CO complex of the remaining heme has its Soret maximum at 447 nm, indicating that the heme still has its native axial thiolate ligand; little conversion to the denatured ‘P420’ form has occurred. This indicates that the remaining heme is located in correctly folded oxygenase domains.

The effects on enzyme activity and CaM/Ca+2 control of G 1074Y and G 1074D nNOS mutations were similar. Although the SI appears well placed to interact with the calmodulin binding region, the calcium dependence of activation is unchanged. However, the NO synthase activity of both mutants, based on heme content as measured at 447 nm in the CO difference spectrum, is only about half that of the wild type enzyme. The activities of the mutants are summarized in Table 1.

TABLE 1

| | | Nitrite |
|---|---|---|
| | | production |
| | Heme/flavin | nmoles/min/mg |
| Enzyme | ratio | NOS |
| wild-type nNOS | 1:1.9 | 292.2 |
| G1074D mutant | 1:2.1 | 179.8 |
| G1074Y mutant | 1:4.0 | 186.2 |
| TAL Deletion | 1:2.4 | 312.3 |
| mutant | | |

Deletion of half of the SI (TAL corresponding to 1072-1074 in nNOS) produced an effect on the spectral properties of the expressed protein analogous to the substitution of Tyr for Gly; in this case the heme/flavin ratio is reduced only to about 1:2.4 (Table 1) (spectrum not shown). The calcium dependence was unaltered. Unlike the Tyr mutant, this mutant had wild type activity on a per heme basis. Deletion of the entire SI produced a spectrally similar nNOS mutant with low activity on a per heme basis.

Cytochrome c reduction by NOS isoforms has been assumed to occur primarily through FMN; recently conducted experiments with an nNOS shielding residue mutant lacking FMN support this view (unpublished). Cytochrome c reduction is therefore a measure of electron transfer through the reductase domains, which does not require the oxygenase component. It is activated by calmodulin binding, but does not strictly correlate with NO production. The cytochrome c reduction experiments presented here correspond to the low salt experiments of Knudsen et al (Knudsen G. M., et al., (2003) J. Biol. Chem., Vol. 278, 31814-31824). ENOS wild type and mutant activity depends on salt concentration and type, and on the buffer used in assays.

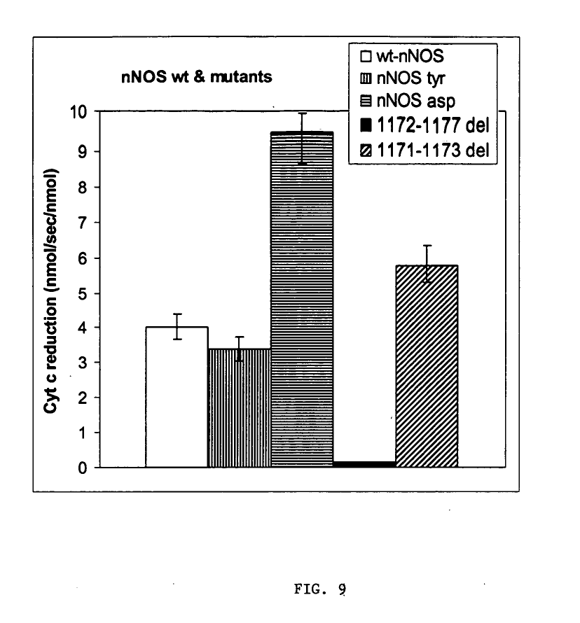

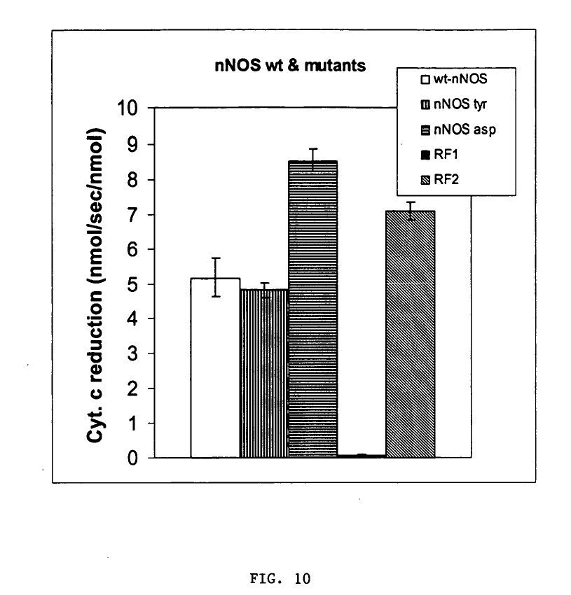

The effects of SI mutations on cytochrome c reduction rates in nNOS are shown in FIGS. 9 and 10. The G1074Y mutant has a slightly reduced rate of cytochrome c reduction consistent with its lower rate of NO production. This is also true of the half deletion mutant. This suggests that the low rate of NO production is the result of slow electron delivery. The enhancement of cytochrome c reduction by calmodulin is comparable (about 5 fold) in wild type nNOS and all mutants except the full SI deletion, which has very low cytochrome c reduction activity. This is in sharp contrast to AI mutants, which tend to lose CaM sensitivity. The G1074D mutant and the 1171-1173 deletion mutants are significantly more active than the wild type enzyme in cytochrome c reduction.

In contrast to nNOS, the SI in eNOS consists entirely of proline and glycine residues unless the flanking residue serine 838 is considered. The eNOS SI was less tolerant of mutations than the nNOS SI, probably because of rigidity imposed by the prolines. In general, 20% of wild type expression levels would produce more than enough enzyme for experiments as described herein, but most of the mutants produced only about 10% heme-containing protein as judged by the small peak in the Soret region; we could detect no CO difference spectra in these preparations at 450 nm (cysteinyl ligand intact) or 420 nm (denatured form).

The G841 deletion mutant, however, was expressed at only slightly lower levels than wild type eNOS (50-60%). UV-visible absorbance spectra of preparations partially purified by 2′5′ affinity chromatography suggested a slightly depressed heme/flavin ratio but were otherwise normal. The Ca+2/CaM dependence of this mutant is unaltered compared to wild type eNOS. The activity per heme is slightly lower in the fully activated state (85% of wild type), which is within the variability of the activity of wild type preparations.

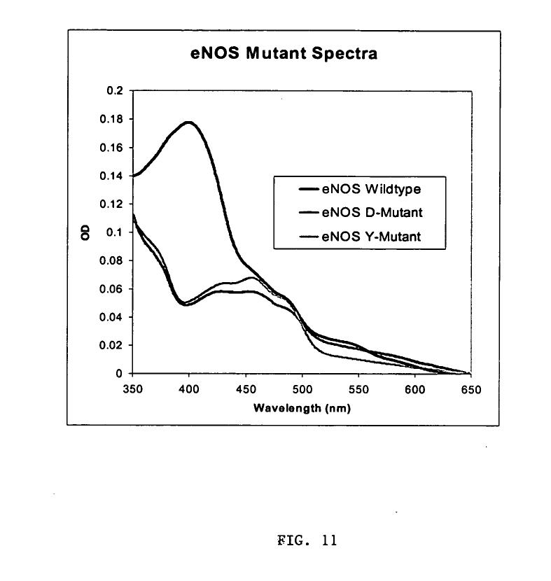

Neither the G841Y nor G841E mutants of bovine eNOS were significantly expressed as heme proteins, in contrast to their nNOS cognates. The heme content measured spectrally in the Soret indicates that only about 10% of the expressed protein binds heme, and we could not observe a CO difference spectrum. The lower heme to flavin ratio observed in G1074Y rat nNOS was reflected in an exaggerated form in the eNOS mutants, which could be partially purified as flavoproteins on a 2′5′ ADP column. The optical spectra of these preparations are compared to wild type eNOS in FIG. 11. The reductase domains of these mutants are obviously inactive in NO production, but exhibit uncoupled NADPH oxidation at two to three times the rate of wild type eNOS.

As previously mentioned, Knudsen et al. reported the effects of SI deletion in eNOS; calcium dependence was affected, but neither the cytochrome c nor the NO production activities were significantly affected, apart from a modest change in salt dependence. The eNOS mutants produced here (except for the G841 deletion, which is similar to wild type) are calmodulin insensitive in all assays, but this may result from exposure of the CaM binding site to proteolysis rather than loss of control because of the removal or mutation of the SI. The pattern of activity is otherwise similar to that observed in nNOS mutants; the tyrosine mutant has low activity, while the G841D mutant and the G841 deletion have slightly higher than wild type rates of cytochrome c reduction.

The G841D mutant was essentially the same as the G841 deletion on a per flavin basis, although because of nearly complete cleavage it was purified as a reductase domain preparation and the G841 deletion is expressed as holoenzyme. The absolute rates are therefore not directly comparable. The G841Y mutant, also purified as a reductase domain preparation, had low activity and was CaM insensitive, in contrast to the corresponding nNOS construct.

Proteolysis of mutants and iNOS. The low heme content in some mutants suggested that the enzyme was susceptible to proteolysis; in purified eNOS and nNOS the exposed calmodulin binding site is the most sensitive in these enzymes to proteolytic cleavage. Since the 2′5′ADP affinity site is associated with the reductase domains, in vivo cleavage at the CaM site would produce flavin bearing reductase domains without heme.

INOS holoenzyme, which lacks the small insertion, cannot be purified without CaM co-expression (Fossetta, J. D. et al., (1996) FEBS Lett 379 (2): 135-138). When we expressed iNOS without CaM co-expression and fractionated the extract on a 2′5′ADP affinity column, we obtained a yellow flavoprotein preparation spectroscopically indistinguishable from the heme free eNOS mutants. The dominant proteins in 2′5′ADP affinity purified preparations of wild type NOS isoforms are NOS holoenzyme and NOS fragments, in our preparations typically corresponding to at least 50% of the total protein; further column purification with gel filtration or anion exchange produces very pure full length NOS. The yield of full-length enzyme in preparations of the low heme mutants is obviously low.

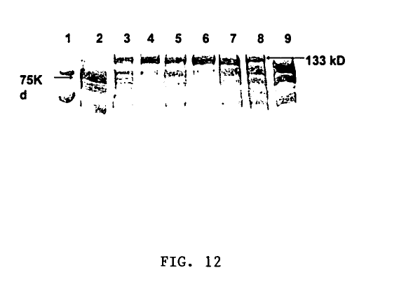

Comparison of SDS-PAGE gels of partially purified wild type iNOS, eNOS and nNOS and selected mutants is shown in FIG. 12. In lane 1 the major band corresponds to nNOS Y879S, an FMN shielding residue mutant which expresses as holenzyme, with a molecular weight of 161 kD (upper left arrow). The nNOS mutants in lanes 2 and 4, which have normal or nearly normal heme content, also are present primarily as holoenzyme. The G1074Y mutant, which was run in lane 3, is approximately half holoenzyme and half flavoprotein fragment (lower left arrow, corresponding to broad bands near 70 kD).

Corresponding wild type eNOS preparations have a major band corresponding to full-length holoenzyme as shown in lane 8 (mW 133 kD; upper center arrow). The eNOS mutants (lanes 9-11) other than the G841 deletion (lane 9) all have their predominant bands at ˜70 kD, corresponding to flavoprotein fragments (lower center arrow). Very little full-length wild type iNOS was observed in lane 6 (upper right arrow); bands at 70 kD corresponding to the flavoprotein reductase fragment can be seen (lower right arrow). It is not clear whether the bands visible near 50 kD are NOS fragments or impurities.

These results indicate that intact holoenzyme is the dominant NOS species in eNOS, nNOS and the heme sufficient mutants. In contrast, iNOS and the low heme mutants have been reduced to reductase fragments by in vivo proteolysis. Neither iNOS nor the heme free mutants have significant bands over 100 kD, and all have multiple bands in the 70 kD region corresponding to proteolytic products cleaved at or near the calmodulin binding site.

Previously identified regions involved in the control of nitric oxide synthesis by Ca+2/CaM have either been calmodulin binding sites or elements involved in suppressing activity in the absence of Ca+2/CaM. The small insertion is clearly correlated with control in the evolution of NOS, and is structurally positioned to interact with established control elements. The information presented here indicate that the role of the SI is complex, and may differ between isoforms. Unlike the AI and the C terminal tail, modification, truncation, or deletion of the small insertion does not consistently result in activation of the enzyme. While AI deletion results in increased activity and reduction of calcium sensitivity, SI modifications result in less straightforward changes in the activity of nitric oxide synthesis.

Typically, AI mutants synthesize NO at low Ca+2 concentrations and have high levels of cytochrome c reductase activity in the absence of calmodulin. In contrast, -SI mutants require CaM for optimal cytochrome c reductase activity as well as NO synthesis, except in cases where the calmodulin binding site has been exposed to cleavage, producing flavoprotein expression. We point out that changes in the calcium dependence of mutants in positions adjacent to the CaM binding site can result from interactions with control elements which function as activators as well as displacement of inhibitors, because these changes merely reflect the necessity of doing work on a protein structural element during CaM binding.

Intentional disruption of the structure of the SI with incompatible substitutions causes significant loss of NO synthesis activity in nNOS, while deletion of a major portion of the SI had a much smaller effect. This suggests that the functions of the SI are ancillary rather than essential for activity; eNOS with an SI deletion or nNOS with a reduced SI can still function, but a disrupted SI can interfere with activation. It is certainly possible that a slightly different full SI deletion in nNOS would also be fully active.

It is noted that wild type NOS enzymes lacking an SI are active as long as they also lack an AI. When we designed these mutants we thought it possible that the AI and SI acted cooperatively, and that the SI might be needed for AI mediated inhibition (e.g., as a lock and clasp). The data presented here instead suggest that the SI may function as an accessory element, since SI mutants may either positively or negatively affect activity.

Our recent proposal of a tethered shuttle mechanism for NOS electron transfer and control (Ghosh, D. K. and Salerno, J. C. (2003) Frontiers in Bioscience 8: D193-D209) provides a context for these observations. In this model, the FMN binding domain shuttles between FAD and heme facing states, both of which bind CaM. CaM facilitates the release of the FMN domain from the reductase complex, where it is in close association with the FAD and NADPH binding domains. The release of the reduced FMN binding domain allows cytochrome c reduction, but is not sufficient to allow NOS production. In holoenzyme, realignment of the FMN binding domain, also CaM facilitated, is necessary for subsequent electron transfer into the oxygenase domain to support catalysis.

The evolutionary ancestors of the NOS reductase domains existed as separate proteins closely related to ferredoxin NADPH reductase and flavodoxin, and in these ancestral electron transfer systems ferredoxin/flavodoxin functioned as a shuttle. The FMN binding domain is essentially a ferredoxin tethered to the two domain reductase unit. In reductase systems in which one component acts as a shuttle, it is common to observe maximum activity at a salt concentration which allows formation of binary complexes for electron transfer (usually optimized at low salt), but does not produce complexes with such slow dissociation rates (optimized at high salt) that the dissociation rate limits the shuttle (Lambeth, J D, et al., H. 1982 Molec. Cell. Biochem. 45:13-31). Salt inhibited shuttles (iNOS) are characterized by relatively weak interactions, while salt stimulated shuttles (eNOS) are characterized by stronger interactions.

The eNOS-SI mutants studied by Knudsen et al. (Knudsen G. M., et al., (2003) J. Biol. Chem., Vol. 278, 31814-31824) were described in terms of SI inhibition which was ‘masked’ by the AI and salt. The -SI and -SI -AI mutants show modestly enhanced cytochrome c reduction with respect to the parent wild type and -AI mutant eNOS enzymes, but only at high KCl; they are slightly slower than their parents at low KCl. Under the conditions which produce enhanced cytochrome c reduction their NO production is lower or at best equal than that of their parents. Enhanced NO production in -SI and -SI -AI mutants with respect to the parent wild type and -AI mutant eNOS enzymes is only observed at low salt; this must be unrelated to the enhanced cytochrome c reduction at high salt. This suggests that steady state cytochrome c reduction by eNOS and all -AI and -SI mutants is limited by the dissociation of a tight complex which is destabilized by the SI.

NO formation is not rate limited by the process which limits cytochrome c reduction. In wild type eNOS it is slightly enhanced at high salt, but in the -SI and -AI -SI mutants NO formation is significantly slower at high salt. This suggests the participation of a complex characterized by weaker interactions in the -SI mutants.

The SI is located in the hinge subdomain, which interacts with all three reductase domains (FMN, FAD and NADPH binding). The position of the SI indicates that it is in direct contact with bound calmodulin, and at the same time other residues in the subdomain are hydrogen bonded both to residues in the other domains and directly to NADP. Although we are not confident enough in the details of models based on incomplete domain structures to assign specific interactions on the FMN binding domain or bound CaM to the SI, its displacement by CaM will clearly affect FMN domain mobility since it forms the terminal of a β hairpin which forms a three stranded β structure with the polypeptide strap linking the FMN and FAD binding domains. In this regard it may serve as an amplifier of CaM driven conformational effects. At the same time, CaM driven conformational effects on the hinge subdomain are likely to be transmitted to the NADPH binding site, linking CaM binding, conformation, and nucleotide binding.

The SI has at least one function indirectly related to control. ENOS and nNOS are resistant to proteolysis in cells even without bound CaM, while iNOS cannot survive in proteolytically deficient E. coli without CaM coexpression. The increased sensitivity of the CaM binding regions in eNOS and nNOS SI mutants imply that the proximity of the SI to the CaM binding region provides some protection to the enzyme from degradation by proteases. It is possible that this is a major function of the SI in cNOS, although it will be necessary to express these mutants in mammalian cells to determine whether the compartmentalization of activities is sufficient to protect the CaM site in SI deficient enzymes.

It is obvious that the protective effect of CaM on iNOS is exerted largely by protecting the protease sensitive CaM binding site. It should be possible to produce intact versions of some mutants which are otherwise produced as reductase fragments by coexpression with CaM, much as iNOS is produced in recombinant systems. In addition, the results presented here suggest the possibility of producing full length iNOS holoenzyme without CaM coexpression by introducing an SI from eNOS or nNOS.

The invention is further illustrated by the following Examples, which are not intended to be limiting in any way.

### EXAMPLE 1

### Identification of Constitutive NOS Activators

It was noted that affinity chromatography purified eNOS is active in the absence of calmodulin prior to dialysis. Removal of eluent 2′ AMP, an NADPH analog, removed CaM independent activity and restored Ca+2/CaM control. 2′ AMP is a competitive inhibitor with respect to NADPH, suggesting that displacement of NADPH/NADP+ from its binding site produced a state in which the reductase domains were competent to support catalysis by electron transfer.

These results were confirmed and extended. Titration of cNOS with 2′AMP not only activated cNOS in the absence of calmodulin, but produced hyperactivation of eNOS in the presence of Ca+2/CaM. Specifically, when the initial concentrations of NADPH and 2′ AMP were equal, eNOS activity was 3-5 times that of eNOS activated by CaM alone. These effects appeared to correlate with changes in the optical spectra of the enzyme in charge transfer region.

These results supported the hypothesis that NADPH/NADP+produces a ‘conformational lock’ in cNOS that inhibits electron transfer (Daff, S. et al., Nitric Oxide 6:366 (2002)). Inhibitors which fill the adenine portion of the pyridine nucleotide binding site can potentially activate electron transfer by displacement of NADPH/NADP+. Since NADPH reduction of cNOS, particularly eNOS, is not close to rate limiting, a wide range of inhibitor concentrations can be tolerated before inhibition of electron donation by NADPH becomes more important than activation by removal of the charge transfer complex. These effects are closely related to loss of control in C terminally truncated cNOS and effects of C terminal phosphorylation (Lane, P. and Gross, S. S., J. Biol. Chem. 277(21): 19087-94 (2002)), which involve adjacent sites.

The initial observations were the result of activity assays of partially purified column fractions. NOS holoenzymes were purified using a 2′5′ADP affinity column; undialyzed eNOS column fractions from this step were essentially calmodulin independent, and were at least 50% as active as purified eNOS preparations (data not shown). The major factors which differentiated the purified control fractions from the undialyzed fractions which has CaM independent activity, were high salt and the presence of an NADPH antagonist, 2′AMP, which was used to elute the column. Although very high ionic strength can partially activate NOS, the salt concentrations associated with the purification were not high enough to produce NOS activation by themselves.

Titration of eNOS with 2′AMP in the presence of ‘fully’ activating levels of Ca+2/CaM was performed using a Greiss assay (see, e.g., the Griess Reagent System, Promega Corporation, Madison, Wis.; and manufacturer's instructions for use). Calcium stoichometries were between two and four. The titration produced an additional activation corresponding to an apparent Kd of ˜1 mM. This corresponded closely to the concentration of NADPH, and suggested that the effect is due to competition of ligands with similar binding constants (NADPH and 2′AMP). In the most widely used assay systems eNOS is 2-3 fold hyperactivated by 2′AMP (Greiss assay). Enhanced levels of cytochrome c reduction (2-5 fold) can also be observed in both eNOS and nNOS.

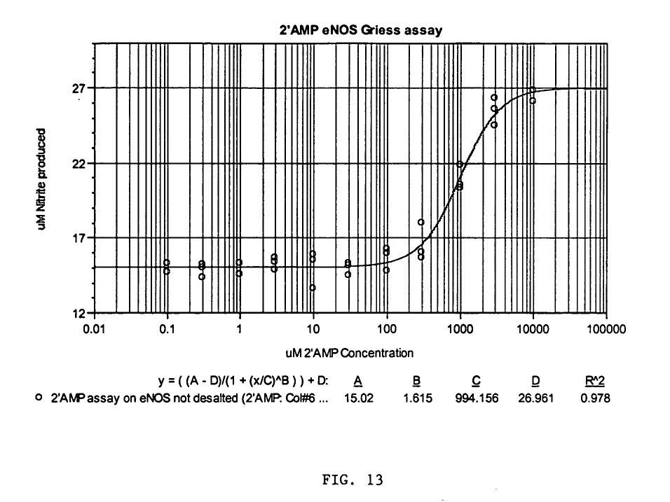

FIG. 13 depicts results from plate reader Greiss assays, demonstrating that the titration of eNOS with 2′AMP in the presence of ‘fully’ activating levels of Ca+2/CaM produced additional activation with apparent Kd of ˜1 mM, suggesting the effect is due to competition of ligands with similar binding constants (NADPH and 2′AMP)

The effect of 2′AMP on the optical spectra of reductase cofactors in eNOS was also examined for eNOS as isolated and reduced with 1 mM NADPH. eNOS only, eNOS NADPH, eNOS NADPH with 2′AMP, eNOS NADPH with 2′AMP and CAM, and NADPH 2′AMP were compared. It was found that sequential addition of 2′AMP and calmodulin caused additional steady state reduction of flavin cofactors, most readily seen below 500 nm, and also a decrease in long wavelength species (data not shown). It was also noted that 2′AMP derivatives functioned as better activators than 3′ derivatives, and AMP-based ligands were more efficient than GMP-based ligands. Cyclic AMP, cyclic GMP, GDP and GTP did not function as activators, and 3′5′ ADP was only a very weak activator.

How do the NOS control elements work together? It is clear that regions of interest are not always close together on the reductase. This is most easily understood in terms of common effects on domain alignment, rather than direct interactions between all the elements involved. In view of the conformation of the enzyme, it is difficult to dock NOS oxygenase domains with a reductase domain model based on NADPH P450 reductase to obtain a close enough distance for electron transfer. A conformational shuttle in which the FMN binding domain moves significantly may be a necessary feature of electron transfer (e.g., Ghosh, D. K. and Salerno, J. C., Front. Biosci. 8:d193-209 (2003)). Multiple constraints may be necessary to interrupt this shuttle.

### EXAMPLE 2

### Confirmation of Activation of NOS by NADPH Analogs

Additional results confirmed the activation of NOS by NADPH analogs and defined conditions under which activation is CaM dependent.

Materials and Methods

All chemicals used for purification were obtained from Sigma Chemical Co. The genes for eNOS and nNOS were gifts from Professor B.S.S. Masters (University of Texas Health Science Center at San Antonio); the iNOS expression system was the gift of Professor Dipak Ghosh (Duke University and VA Medical Center). GroELS plasmid was provided by Dr. Anthony Gatenby (Dupont).

Expression and purification of bovine eNOS and rat nNOS. Expression and purification of bovine eNOS and rat nNOS were performed using procedures similar to those previously described (Martasek, P.,et al., (1996) Biochem Biophys Res Commun. 219(2); 359-65). Transformed cells were broken with a French press, and after centrifugation to remove cell debris the supernatant was loaded on a 2′5′ADP affinity column, washed, and eluted with 2′AMP as described (id). High purity preparations can be obtained with a size exclusion step; we used a Superose 6 HR 10/30 column (Pharmacia Biotech AB, Uppsala, Sweden), flow rate 0.4 mL/min, buffer composition −50 mM Tris-HCl, pH 7.5, 0.1 mM EDTA, 1 mM-mercaptoethanol, 100 mM NaCl, 10% glycerol (vol/vol). During the purification procedure BH4 or L-arginine were added after elution from the affinity column. Enzyme sample concentrations were determined on the basis of heme concentration, except in heme free preparations, where the flavin concentration was measured. UV-visible absorbance spectra were recorded on an Aminco DW-2000 spectrophotometer.

Nitric Oxide production was assayed using the Griess assay as adapted for microtiter plates (Gross, S. S. (1996) Methods Enzymol. 268:159-68); calcium dependence was measured using EDTA as a Ca+2 buffer system. NOS isoforms were routinely assayed with different buffering systems, since eNOS is much more active in MOPS while iNOS and eNOS work well in Tris. NADPH-dependent cytochrome c reduction was measured spectrophotometrically as described by McMillan et al (McMillan, K., et al., (1992) Proc Nat. Acad. Sci. U.S.A. 89, 11141-11145), adapted to 96-well plate microtiter plates. In each well, the 500-uL reaction mixture contained 50 mM Na+ TRIS buffer, pH 7.5, 50 uM EDTA, 50 uM NADPH, 50 uM horse heart cytochrome c, and ˜10 nM of nNOS. Cytochrome c reduction was monitored at 550 nm (ε=2.1×104/M). In Cam/Ca+2 dependence studies, 0.75 uM CaCl₂ and 10 pg/ml calmodulin were added to reaction mixtures. Assays were performed on a SpectraMAX plate reader (Molecular Devices).

Results

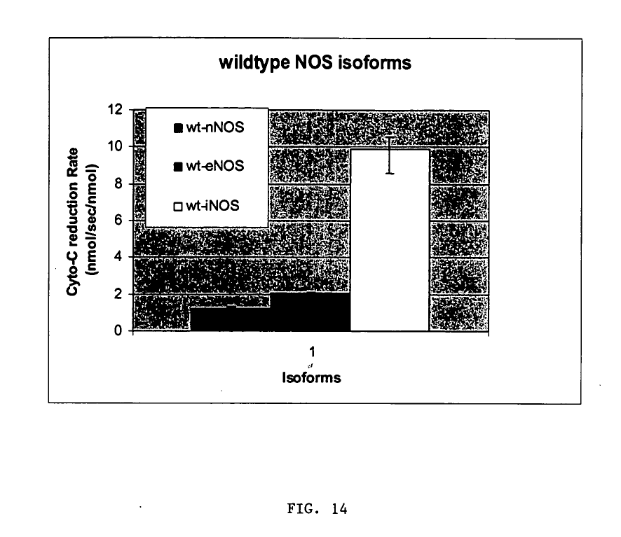

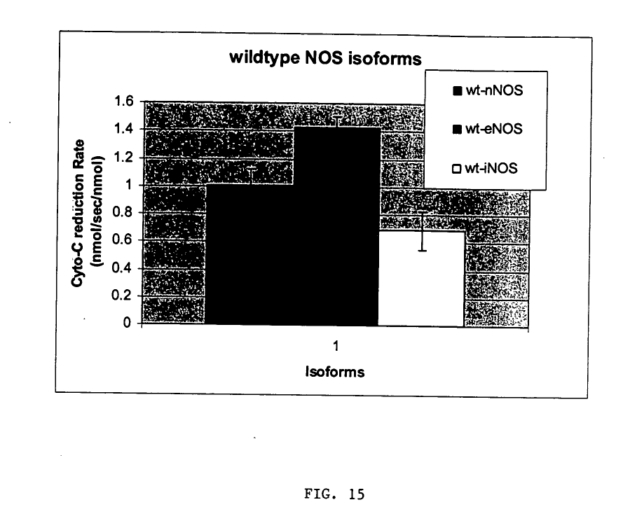

Tris buffer provided an environment in which eNOS is activated in the absence of CaM, providing about 50% of the maximal rate seen in CaM activation. This rate could be increased by NADPH analogs. nNOS is not activated in Tris without CaM. In Good's buffer systems (e.g., HEPES or MOPS), eNOS and nNOS were both well controlled by Ca+2/CaM. In both cases, NADPH analogs did not activate in the absence of CaM, but 2-3 fold hyperactivation of eNOS could be readily obtained with 2′ AMP. FIG. 14 depicts activation of electron transfer wild type cNOS isoforms by ATP in Tris buffer in the absence of calmodulin. FIG. 15 demonstrates that INOS is inhibited under the same conditions.

Cytochrome c reduction experiments indicated that cNOS (eNOS and nNOS) activation by NADPH analogs included at least 2 fold activation of cytochrome c reduction as well as NO. synthesis. Without being bound by a particular theory, it is believed that a major component of activation by NADPH analogs was the release of the locked closed reductase conformation. This was consistent with the finding that NADPH analogs inhibited iNOS at high concentrations, indicative of binding to the NADPH site, but did not activate it at any concentration, because iNOS reduction of cytochrome c is independent of CaM. The closed reductase of iNOS appeared to freely dissociate, and iNOS utilizes CaM only for the association of the FMN binding domain with the oxygenase domain.

The teachings of all references cited are hereby incorporated herein in their entirety. While this invention has been particularly shown and described with references to preferred embodiments thereof, it will be understood by those skilled in the art that various changes in form and details may be made therein without departing from the spirit and scope of the invention as defined by the appended claims.

1. An agent which activates a constitutive nitric oxide synthase by inhibitor displacement.

2. An agent of claim 1, wherein the agent displaces NADP+/NADPH.

3. An agent of claim 1, wherein the agent is an agent which has binding domain overlap on the nitric oxide synthase with NADPH.

4. An agent of claim 3, wherein the agent fills the adenine portion of the pyridine nucleotide binding site, but does not initiate inhibition of electron transfer.

5. An agent of claim 1, wherein the agent prevents binding of NADPH to the nitric oxide synthase.

6. An agent of claim 1, wherein the agent comprises a heterocyclic aromatic ring having one or more side chains upon which one or more negatively charged atoms or molecules is attached.

7. A composition comprising an agent of claim 1.

8. A composition of claim 7, wherein the composition comprises a physiologically acceptable carrier.

9. A composition of claim 7, wherein the composition further comprises other therapeutic agents.

10. A method of altering activity of a constitutive nitric oxide synthase, comprising contacting the nitric oxide synthase with an agent of claim 1.

11. A method of claim 10, wherein the constitutive nitric oxide synthase is endothelial nitric oxide synthase.

12. A method of claim 10, wherein the constitutive nitric oxide synthase is neuronal nitric oxide synthase.

13. A method of altering activity of a constitutive nitric oxide synthase, comprising contacting the nitric oxide synthase with an NADPH analog.

14. A method of claim 13, wherein the NADPH analog is selected from the group consisting of: 2′ AMP, 5′ AMP, 2′5′ADP, ADP and ATP.

15. A method of treating a disease modulated by production of nitric oxide by a constitutive nitric oxide synthase in an individual, comprising administering to the individual an effective amount of an agent of claim 1.

16. A method of treating a disease modulated by production of nitric oxide by a constitutive nitric oxide synthase in an individual, comprising administering to the individual an effective amount of an NADPH analog.

17. A method of claim 16, wherein the NADPH analog is selected from the group consisting of: 2′ AMP, 5′ AMP, 2′5′ADP, ADP and ATP.

18. A method of identifying an agent that modulates activity of a constitutive nitric oxide synthase, comprising assaying the ability of the agent to displace an inhibitor.

19. A method of identifying an agent that modulates activity of a constitutive nitric oxide synthase, comprising assaying the ability of the agent to compete with NADPH for binding to the constitutive nitric oxide synthase.

20. A method of altering activity of a constitutive nitric oxide synthase, comprising contacting the nitric oxide synthase with an agent of claim 1, wherein the agent is incorporated into a biocompatible carrier.

21. The method of claim 20, wherein the biocompatible carrier is coated on or part of an implantable medical device.

22. The method of claim 21, wherein the implantable medical device is a stent.

23. A method of claim 20, wherein the agent of claim 1 is an NADPH analog, and the NADPH analog is selected from the group consisting of: 2′ AMP, 5′ AMP, 2′5′ADP, ADP and ATP.


SEQUENCE LISTING (17 sequences)

SEQ ID NO: 1 - DNA, 56 bp, rattus
ggtggagatgctggaggagaggaacactgctctgggtgtcatcagtaattggaagg

SEQ ID NO: 2 - DNA, 53 bp, bovine
tgtggagcagctggagaaaggcagcccaggcggccctcctcccagctgggtgc

SEQ ID NO: 3 - DNA, 38 bp, mus musculus
cctggaggacctggatgagagtggcagctactgggtca

SEQ ID NO: 4 - protein, 19 aa, rattus
VEMLEERNTALGVISNWKD

SEQ ID NO: 5 - protein, 18 aa, bovine
VEQLEKGSPGGPPPSWVR

SEQ ID NO: 6 - protein, 13 aa, mus musculus
LEDLDESGSYWVK

SEQ ID NO: 7 - protein, 12 aa, hom sapiens
LNNLDEESNKKH

SEQ ID NO: 8 - protein, 4 aa, homo sapiens
GVIS

SEQ ID NO: 9 - protein, 7 aa, homo sapiens
TALYVIS

SEQ ID NO: 10 - protein, 7 aa, homo sapiens
TALDVIS

SEQ ID NO: 11 - protein, 7 aa, homo sapiens
SPGGPPP

SEQ ID NO: 12 - protein, 6 aa, homo sapiens
SPGPPP

SEQ ID NO: 13 - protein, 7 aa, homo sapiens
SPGYPPP

SEQ ID NO: 14 - protein, 7 aa, homo sapiens
SPGDPPP

SEQ ID NO: 15 - protein, 5 aa, homo sapiens
LDEES

SEQ ID NO: 16 - protein, 7 aa, homo sapiens
TALGVIS

SEQ ID NO: 17 - protein, 6 aa, homo sapiens
PGGPPP